(19) United States
(12) Patent Application Publication
(10) Pub. No.: US 2024/0122947 A1
(43) Pub. Date: Apr. 18, 2024

(54) A CANNABINOID MIXTURE

(71) Applicants: TTS Pharma Ltd, Liverpool, Merseyside (GB); Aberystwyth University, Aberystwyth, Ceredigion (GB)

(72) Inventors: Robert WALTON, Liverpool, Merseyside (GB); Mark TUCKER, Liverpool, Merseyside (GB); Debbie NASH, Aberystwyth, Ceredigion (GB); Nathan ALLEN, Aberystwyth, Ceredigion (GB); Ruth WONFOR, Aberystwyth, Ceredigion (GB); Ifat SHAH, Aberystwyth, Ceredigion (GB)

(73) Assignees: TTS Pharma Ltd, Liverpool, Merseyside (GB); Aberystwyth University, Aberystwyth, Ceredigion (GB)

(21) Appl. No.: 18/276,200

(22) PCT Filed: Feb. 8, 2022
(86) PCT No.: PCT/GB2022/050334

§ 371 Date: Aug. 7, 2023

(30) Foreign Application Priority Data
Feb. 8, 2021 (GB) 2101732.2

(51) Int. Cl. A61K 31/00; A61P 29/00

(52) U.S. Cl. CPC A61K 31/658; A61P 29/00

(57) ABSTRACT

There is disclosed a composition comprising cannabidiol (CBD) and one or more cannabinoids selected from cannabidiolic acid (CBDA), cannabigerol (CBG) and cannabigerolic acid (CBGA) for use in the treatment or prevention of inflammation and disorders associated with inflammation or an IL-6 related disorder.

### FIELD OF INVENTION

The present invention provides a composition comprising cannabidiol (CBD) and one or more cannabinoids selected from cannabidiolic acid (CBDA), cannabigerol (CBG) and cannabigerolic acid (CBGA) for use in a therapeutic treatment. The present invention also relates to a food or beverage product comprising such a composition.

### BACKGROUND

Cannabinoid compositions, and the uses thereof, are a growing area of study and commercial activity.

Commercially available products include, for example, a broad-spectrum cannabinoid oil, which is available as a 5%, 10%, 20% and 40% w/w cannabidiol (CBD) oil (https://www.budandtender.com/pages/certificate-of-analysis-bto05-0010, https://www.budandtender.com/pages/certificate-of-analysis-bto10-0010, https://www.budandtender.com/pages/certificate-of-analysis-bto20-0013 and https://www.budandtender.com/pages/certificate-of-analysis-bto40-0011, respectively). None of the 5%, 10%, 20% or 40% w/w CBD oils contain cannabidiolic acid (CBDA).

Marx et al. (2018, Journal of Toxicology, Article ID 8143582) provides a supercritical CO2 extract of the aerial parts of the Cannabis sativa plant, which is 26% w/w phytocannabinoids and 61% edible fatty acids, with the remaining 13% including fatty alkanes, plant sterols, triterpenes and tocopherols. The aim of this study was to perform toxicological studies on the extract, and no uses of the extract are disclosed.

Radmila et al. (2018, Molecules, 23, 1230) provides an analysis of 15 commercially available CBD oils. Of these, Oil 3 was found to comprise 0.79% w/w (3247 μg/g) CBD, 16 μg/g cannabigerol (CBG), 174.05 μg/g total terpenes and 148 μg/g tetrahydrocannabinol (THC), with the carrier oil being olive oil. Oil 12 was found to comprise 1.61% w/w (12,758 μg/g) CBD, 6 μg/g CBG, 981.37 μg/g total terpenes and 494 μg/g THC, with the carrier oil being hemp seed oil. Oil 14 was found to comprise 3.09% w/w (23,186 μg/g) CBD, 460 μg/g CBG, 752.82 μg/g total terpenes and 524 μg/g THC, with the carrier oil being hemp seed oil.

With regard to the use of cannabinoid compositions in the treatment of inflammation or disorders associated with inflammation, there are two reports that Nabiximols (trade name Sativex) has an analgesic effect in patients with Rheumatoid Arthritis (Blake et al. 2006, Rheumatology; 45:50-52 and Richards et al. 2012, Cochrane Database of Systematic Reviews; Issue 1, Art No. CD008921). Sativex is provided as an oromucosal spray, with each spray delivering a dose of 2.7 mg tetrahydrocannabinol (THC) and 2.5 mg cannabidiol (CBD).

Despite the existence of the cannabinoid compositions mentioned above, there remains a need for new cannabinoid compositions, especially new cannabinoid compositions with improved activity.

### SUMMARY OF INVENTION

The present invention arises from the surprising finding that a cannabinoid composition containing cannabidiol (CBD) and one or more cannabinoids selected from cannabidiolic acid (CBDA), cannabigerol (CBG) and cannabigerolic acid (CBGA) is particularly effective in the treatment of inflammation or a disorder associated with inflammation.

Accordingly, in one aspect of the invention there is provided a composition comprising cannabidiol (CBD) and cannabidiolic acid (CBDA), for use in the treatment or prevention of inflammation, or a disorder associated with inflammation.

According to another aspect of the invention there is provided a composition comprising cannabidiol (CBD) for use in the treatment or prevention of inflammation, a disorder associated with inflammation or a IL-6-related disorder.

According to another aspect of the present invention there is provided a composition comprising cannabidiol (CBD) and one or more cannabinoids selected from cannabidiolic acid (CBDA), cannabigerol (CBG) and cannabigerolic acid (CBGA) for use in the treatment or prevention of inflammation, a disorder associated with inflammation, or an IL-6-related disorder.

Conveniently, the inflammation or disorder associated with inflammation is associated with increased Pattern Recognition Receptor (PRR) activation or increased interleukin-6 (IL-6) production.

Preferably, the Pattern Recognition Receptor (PRR) is a Toll-like Receptor.

Advantageously, the inflammation or disorder associated with inflammation is selected from rheumatoid arthritis, juvenile idiopathic arthritis, ankalosing spondylitis, takayasu arteritis, giant cell arteritis, atherosclerosis, intracerebral haemorrhage, Behcet's disease, polymyalgia rheumatic, familial Mediterranean fever, thyroid eye disease, systemic lupus erythematosus, endometritis, inflammatory bowel disease, equine gastric ulcer syndrome, acute respiratory distress syndrome, asthma, cystitis, ulcerative colitis, Crohn's disease, cancer, Castleman disease, infectious diseases such as COVID-19, encephalitis, enterovirus 71 and hand foot and mouth disease, severe asthma, psychiatric illnesses such as depression, schizophrenia and diseases related to epigenetic modification, neurological diseases such as neuromyelitis optica, multiple sclerosis and Alzheimer's disease, and dermatological diseases such as eczema, atopic dermatitis, psoriasis and acne, diabetes, obesity and ageing.

Preferably, the inflammatory bowel disease is selected from ulcerative colitis and Crohn's disease.

Preferably, the cancer is selected from Castleman disease, multiple myeloma, prostate cancer, breast cancer, oral cancer, metastatic cancer, pancreatic cancer, lung cancer, liver cancer, bladder cancer, Waldenstroms macroglobulinaemia, vulvar cancer and kaposis sarcoma.

Conveniently, the composition further comprises at least one terpene.

Preferably, the terpene:cannabinoid ratio by weight is 1:3 to 1:60 w/w. more preferably 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:9 to 1:20 w/w, or 1:15 to 1:20 w/w.

Preferably, the at least one terpene is terpinolene.

Advantageously, the at least one terpene comprises alpha-humulene, beta-caryophyllene, alpha-pinene, beta-pinene, terpinolene, beta myrcene and/or an ocimene isomer.

Advantageously, the total concentration of CBD and CBDA is 0.03-30% w/w, preferably 1-25% w/w, more preferably 5-25% w/w.

Conveniently, the total cannabinoid is present at a concentration of 0.03-30% w/w, preferably 1-25% w/w, more preferably 5-25% w/w.

Preferably, the at least one terpene is present at a concentration of 0.01-10% w/w, preferably 0.1-6% w/w, more preferably 0.5-3% w/w.

Preferably, the composition further comprises cannabigerol (CBG) and/or cannabidivarin (CBDV).

Conveniently, the CBDV:CBD ratio by weight is 1:10 to 1:30 w/w, preferably 1:15 to 1:25 w/w, more preferably 1:20 to 1:25 w/w.

Preferably, the CBDA:CBD ratio by weight is from 1:2 to 1:20 w/w, more preferably 1:2 to 1:15 w/w, more preferably 1:3 to 1:10 w/w, more preferably 1:4 to 1:6 w/w.

Advantageously, the composition further comprises an oil.

Conveniently, the cannabinoid:oil ratio is 1:2 to 1:3500 w/w.

Preferably, the oil is present at 60-99.96% w/w.

Advantageously, the oil is selected from hemp seed oil, rape seed oil, coconut oil and MCT oil, or a mixture or two or more oils selected from hemp seed oil, rape seed oil, coconut oil and MCT oil.

According to another aspect of the present invention, there is provided a food or beverage product comprising a composition of the invention for use in the treatment or prevention of inflammation or a disorder associated with inflammation.

According to a further aspect of the present invention, there is provided the use of the composition of the invention for the treatment or prevention of non-clinical inflammation.

In this specification the term “cannabinoid” means chemicals that are found in Cannabis plants. The term includes cannabinoids found in plants other than Cannabis plants, such as Echinacea purpurea, Echinacea angustifolia, Acemila oleracea, Helichrysum umbraculigerum and Radula marginata. The term includes both phytocannabinoids and synthetic cannabinoids. It includes the classes of cannabidiol (CBD) and cannabigerol (CBG). The term “cannabinoid” also includes the classes of cannabichromene, cannabicyclol, cannabivarin, tetrahydrocannabivarin, cannabidivarin, cannabichromevarin, cannabigerovarin, cannabigerol monomethyl ether, cannabielsoin and cannabicitran. The term “cannabinoid” also covers modified versions of the naturally occurring cannabinoids which retain at least 20% of the activity. The term “cannabinoid” also includes controlled cannabinoids such as trans-delta-9-tetrahydrocannabinol-C5, Cis-delta-9-tetrahydrocannabinol-C5, Delta-9-tetrahydrocannabinol-C4, Delta-9-tetrahydrocannabinol-C3 (Delta-9-tetrahydrocannabivarin), Delta-9-tetrahydrocannabinol-C1, Delta-8-tetrahydrocannabinol, Cannabinol-C1, Cannabinol-C2, Cannabinol-C3, Cannabinol-C4, Cannabinol-C5 and Cannabinol methyl ether-C5.

In this specification the term “treatment” means complete cure of a clinical or non-clinical condition as well as partially alleviating the symptoms thereof but without complete cure of the condition. It also refers to both short-term alleviating of symptoms as well as long term alleviating of symptoms.

In this specification the term “prevention” means complete prevention of a clinical or non-clinical condition as well as the partial prevention of symptoms thereof but without complete prevention of the condition. It also refers to both short-term prevention of symptoms as well as long term prevention of symptoms.

In this specification the term “CBDA:CBD ratio” is a ratio of the total amount (w/w) of CBDA present in the composition to the total amount (w/w) of CBD present in the composition.

In this specification, the term “CBDV:CBD ratio” is a ratio of the total amount (w/w) of CBDV present in the composition to the total amount (w/w) of CBD present in the composition.

In this specification the term “terpene:cannabinoid ratio” is a ratio of the total amount (w/w) of all terpenes present in the composition to the total amount (w/w) of cannabinoids present in the composition. However, it will be appreciated that in some embodiments of the present invention, the “terpene:cannabinoid ratio” refers to the ratio of the total amount (w/w) of the following terpenes present in the composition (alpha-humulene, beta-caryophyllene, alpha-pinene, beta-pinene, terpinolene, beta myrcene and trans beta ocimene) to the total amount (w/w) of cannabinoids present in the composition.

In this specification the term “ocimene isomer” refers to any of alpha-ocimene, trans beta-ocimene and cis beta-ocimene.

In this specification the term “cannabinoid:oil ratio” is a ratio of the total amount (w/w) of cannabinoids present in the composition to the total amount (w/w) of carrier oils present in the composition.

### BRIEF DESCRIPTION OF THE FIGURES

FIG. 1 shows the results of an enzyme-linked immunosorbent assay (ELISA) showing the IL-6 concentration in response to the addition of LPS and/or crude hemp extract (Fraction 2). From left to right=Sample 1—media only; Sample 2-10 μg hemp crude extract; Sample 3-25 μg hemp crude extract; Sample 4-50 μg hemp crude extract; Sample 5—LPS only; Sample 6—LPS+10 μg hemp crude extract; Sample 7—LPS+25 μg hemp crude extract; Sample 8—LPS+50 μg hemp crude extract. LPS was administered at 500 ng.

FIGS. 2 and 3 show the results of an enzyme-linked immunosorbent assay (ELISA) showing the IL-6 or PGF_{2α} concentration in response to the addition of LPS and/or crude hemp extract (Fraction 2). From left to right=Sample 1—media only; Sample 2—10 μg/mL hemp crude extract; Sample 3-25 μg/mL hemp crude extract; Sample 4—50 μg/mL hemp crude extract; Sample 5—LPS only; Sample 6—LPS+10 μg/mL hemp crude extract; Sample 7—LPS+25 μg/mL hemp crude extract; Sample 8—LPS+50 μg/mL hemp crude extract. LPS was administered at 500 ng/mL.

FIGS. 4 to 6 show the results of an enzyme-linked immunosorbent assay (ELISA) showing the IL-6, PGF_{2α} or PGE₂ concentration in response to the addition of LPS and/or the purified CBG/CBD compound (50 μg/mL CBG: 20 μg/mL CBD). From left to right=Sample 1—media only; Sample 2-10 μg/mL CBG/CBD compound; Sample 3—25 μg/mL CBG/CBD compound; Sample 4-50 μg/mL CBG/CBD compound; Sample 5—LPS only; Sample 6—LPS+10 μg/mL CBG/CBD compound; Sample 7—LPS+25 μg/mL CBG/CBD compound; Sample 8—LPS+50 μg/mL CBG/CBD compound. LPS was administered at 500 ng/mL.

FIG. 7 shows UV chromatograms of Fractions 1 to 3.

FIG. 8 shows a UV chromatogram of Fraction 2.

FIG. 9 shows UV chromatograms of Fractions 1 to 3.

FIG. 10 shows MS2 chromatograms of Fractions 1 to 3. Unidentified compound.

FIG. 11 shows a UV spectrum of Fraction 2. Unidentified compound.

FIG. 12 shows MS chromatograms of Fractions 1 to 3. Unidentified compound.

FIG. 13 shows chromatograms of Fractions 1 to 3. Compound identified as cannflavin A.

### DETAILED DESCRIPTION

In general terms, the present invention relates to a composition comprising cannabidiol (CBD) and one or more cannabinoids selected from cannabidiolic acid (CBDA), cannabigerol (CBG) and cannabigerolic acid (CBGA), and optionally further components such as a terpene and/or a carrier oil, for use in the treatment or prevention of inflammation, a disorder associated with inflammation or an IL-6 related disorder. Specific embodiments of the components of the composition will now be described.

Cannabinoid

In the present invention, the composition comprises cannabidiol (CBD). In particular, the composition comprises CBD and one or more cannabinoid selected from cannabidiolic acid (CBDA), cannabigerol (CBG) and cannabigerolic acid (CBGA), for example, CBD and CBDA. In some embodiments one or more of the cannabinoids is provided as an extract or extracts from a Cannabis plant, preferably of the species Cannabis sativa or Cannabis indica, whereas in other embodiments one or more of the cannabinoids is provided as a synthetic compound or a mixture of synthetic compounds, or as a chemically modified compound or mixture of chemically modified compounds. In other embodiments, one or more of the cannabinoids is provided as an extract from species of plant other than Cannabis plants, such as Echinacea purpurea, Echinacea angustifolia, Acemila oleracea, Helichrysum umbraculigerum, and Radula marginata.

In some embodiments, one or more of the cannabinoids is extracted from the Cannabis plant and the extract is further refined using a further stage of processing (for example, crystallisation technologies) which results in a crystallised, isolated cannabinoid, which is also referred to as an isolate or co-crystallised isolate. In some embodiments, the cannabinoid isolate has enhanced solubility and performance characteristics compared with the cannabinoid extract.

In some embodiments, the composition comprises CBD and CBDA. In some embodiments, the composition further comprises cannabigerol (CBG) and/or cannabidivarin (CBDV).

Cannabidiol (CBD), shown in formula (i) below, is a cannabinoid with the chemical formula C₂₁H₃₀O₂ and a molecular weight of 314.469 g/mol. The term “cannabidiol” also covers variants of CBD with different lengths of the alkyl chain, which vary in length from 1-5 carbons (and therefore the molecular weight of CBD varies from 266.426 g/mol to 314.469 g/mol). Depending on growing conditions it can constitute up to 40% of the extracts of the Cannabis sativa plant. CBD is not psychoactive or hallucinogenic.

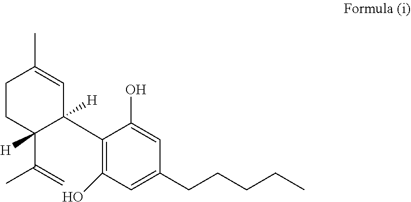 [SMILES: C=C(C)[C@@H]1CCC(C)=C[C@H]1c1c(O)cc(CCCCC)cc1O]

Cannabidiolic acid (CBDA), shown in formula (ii) below, is a cannabinoid with the chemical formula C₂₂H₃₀O₄ and a molecular weight of 358.5 g/mol. The term “cannabidiolic acid” also covers variants of CBDA with different lengths of the alkyl chain. In cannabinoid biosynthesis, CBDA is a precursor to CBD. In particular, during cannabinoid biogenesis, cannabigerolic acid (CBGA) is converted to CBDA, and CBDA is then converted into CBD through decarboxylation, whereby CBDA loses its acidic carboxyl group. Like CBD, CBDA is not psychoactive or hallucinogenic.

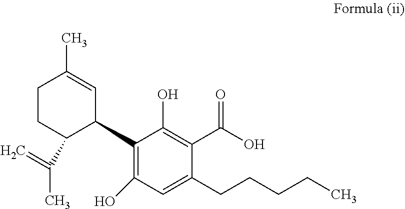 [SMILES: C=C(C)[C@@H]1CCC(C)=C[C@H]1c1c(O)cc(CCCCC)c(C(=O)O)c1O]

Cannabigerol (CBG), shown in formula (iii) below, is a cannabinoid with the chemical formula C₂₁H₃₂O₂ and a molecular weight of 316.485 g/mol. The term “cannabigerol” also covers variants of CBG with different lengths of the alkyl chain. Like CBD, CBG is not psychoactive or hallucinogenic. In some embodiments, the composition in total comprises ≤0.1% w/w, preferably ≤0.01% w/w, more preferably ≤0.001% w/w CBG.

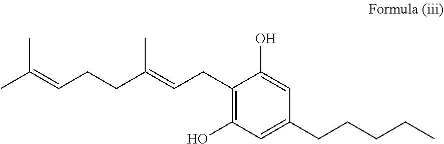 [SMILES: CCCCCc1cc(O)c(C/C=C(\C)CCC=C(C)C)c(O)c1]

Cannabigerolic acid (CBGA), shown in formula (iv) below, is a cannabinoid with the chemical formula C₂₂H₃₂O₄ and a molecular weight of 360.5 g/mol. The term “cannabidiolic acid” also covers variants of CBGA with different lengths of the alkyl chain. In cannabinoid biosynthesis, CBGA is a precursor to CBG and CBDA. In particular, during cannabinoid biogenesis, cannabigerolic acid (CBGA) is converted into CBG through decarboxylation, whereby CBGA loses its acidic carboxyl group. Like CBG, CBGA is not psychoactive or hallucinogenic.

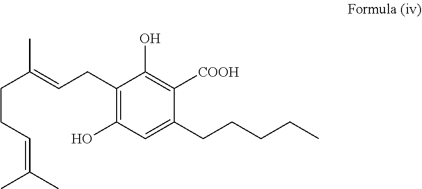 [SMILES: CCCCCc1cc(O)c(C/C=C(\C)CCC=C(C)C)c(O)c1C(=O)O]

Cannabidivarin (CBDV), shown in Formula (v) below, is a cannabinoid with the chemical formula C₁₉H₂₆O₂ and a molecular weight of 286.415 g/mol. CBDV is a non-psychoactive cannabinoid.

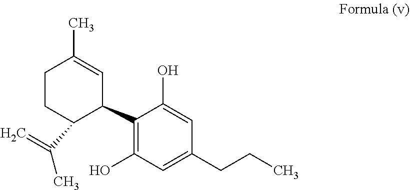 [SMILES: C=C(C)[C@@H]1CCC(C)=C[C@H]1c1c(O)cc(CCC)cc1O]

In some embodiments, the CBDA:CBD ratio by weight is from 1:2 to 1:20 w/w, preferably from 1:2 to 1:15 w/w, more preferably 1:3 to 1:10 w/w, further preferably 1:4 to 1:6 w/w.

In some embodiments, the CBDV:CBD ratio by weight is 1:15 to 1:30 w/w, preferably 1:15 to 1:25 w/w, more preferably 1:20 to 1:25 w/w.

Tetrahydrocannabinol (THC; C₂₁H₃₀O₂), also known as delta-9-Tetrahydrocannabinol or (−)-trans-Δ⁹-Tetrahydrocannabinol, Cannabinol (CBN; C₂₁H₂₆O₂) and tetrahydrocannabinolic acid (THC-A; C₂₂H₂₀O₄) are psychoactive cannabinoids found in Cannabis plants, including the Cannabis sativa and Cannabis indica plants. In some embodiments, the composition in total comprises s 0.001% w/w of 9-tetrahydrocannabinol (THC).

Terpenes

In some embodiments, the composition comprises at least one terpene.

In some embodiments, the at least one terpene is terpinolene. In some embodiments, the composition further comprises one or more terpenes from: alpha-humulene, beta-caryophyllene, alpha-pinene, beta-pinene, beta-myrcene (also known as myrcene) and/or an ocimene isomer. In preferred embodiments of the present invention, the composition comprises beta-caryophyllene, alpha-humulene, alpha-pinene, beta-pinene, terpinolene, beta-myrcene and an ocimene isomer.

In some embodiments the at least one terpene is provided as an extract from a plant or insect. In some embodiments the at least one terpene is provided as an extract or extracts from a Cannabis plant, preferably of the species Cannabis sativa or Cannabis indica, whereas in other embodiments the at least one terpene is provided as a synthetic compound or a mixture of synthetic compounds, or as a chemically modified compound or mixture of chemically modified compounds.

In some embodiments, the composition consists essentially of CBD and CBDA. In other embodiments, the composition consists essentially of CBD, CBDA and a terpene selected from: beta-caryophyllene, alpha-humulene, alpha-pinene, beta-pinene, terpinolene, beta-myrcene, an ocimene isomer and mixtures thereof. In preferred variants of this embodiment, the terpene:cannabinoid ratio is 1:3 to 1:60 w/w, preferably 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:9 to 1:20 w/w, or 1:15 to 1:20 w/w.

In some embodiments, the composition consists essentially of CBD and one or more cannabinoid selected from CBDA, CBG and CBGA. In other embodiments, the composition consists essentially of CBD, one or more cannabinoid selected from CBDA, CBG and CBGA, and a terpene selected from: beta-caryophyllene, alpha-humulene, alpha-pinene, beta-pinene, terpinolene, beta-myrcene, an ocimene isomer and mixtures thereof. In preferred variants of this embodiment, the terpene:cannabinoid ratio is 1:3 to 1:60 w/w, preferably 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:9 to 1:20 w/w, or 1:15 to 1:20 w/w.

In some embodiments, the composition comprises beta-caryophyllene, alpha-humulene, alpha-pinene, beta-pinene, terpinolene, beta-myrcene, an ocimene isomer and a least one other sesquiterpene.

In preferred embodiments, the ocimene isomer is trans beta ocimene.

Terpenes are a large and diverse group of organic compounds, in particular hydrocarbons, produced by a variety of plants, including Cannabis sativa and Cannabis indica. Terpenes are classified by the number of isoprene units that are present in the molecule; for example, monoterpenes consist of two isoprene units and have the molecular formula C₁₀H₁₆. Limonene and pinene, for which there are two structural isomers alpha-pinene and beta-pinene, are examples of monoterpenes. Triterpenes consist of six isoprene units and have the formula C₃₀H₄₈. Squalene is an example of a triterpene. Sesquiterpenes consist of three isoprene units and have the formula C₁₅H₂₄. Humulene, which is also known as alpha-humulene, and beta-caryophyllene are examples of sesquiterpenes.

The structures of two terpenes, alpha-humulene (a sesquiterpene; formula (vi)) and terpinolene (a monoterpene; formula (vii)), are shown below.

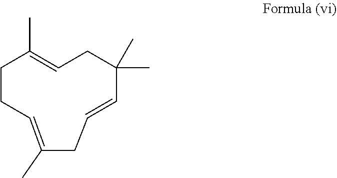

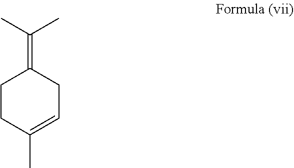

The profile of terpenes present in the composition contributes to the flavour and smell of the composition.

Terpenoids are modified terpenes that contain additional functional groups. In some embodiments of the present invention, the at least one terpene comprises at least one terpenoid.

In embodiments of the present invention, the terpene(s) and cannabinoid(s) are present in the composition in a ratio of 1:3 to 1:60 w/w, preferably 1:3 to 1:40 w/w, 1:3 to 1:35 w/w, 1:3 to 1:20 w/w, 1:5 to 1:20 w/w, 1:5 to 1:9 w/w, 1:5 to 1:7 w/w, 1:7 to 1:12 w/w, 1:9 to 1:20 w/w, or 1:15 to 1:20 w/w.

Flavonoids

In embodiments of the present invention, the composition comprises one or more flavonoids. Flavonoids are produced by a variety of plants and have the general structure of a 15-carbon skeleton, which consists of two phenyl rings and a heterocyclic ring. This carbon structure is sometimes referred to as a C6-C3-C6 structure.

In some embodiments the one or more flavonoids are provided as an extract from a plant. In some embodiments the one or more flavonoids are provided as an extract or extracts from a Cannabis plant, preferably of the species Cannabis sativa or Cannabis indica, whereas in other embodiments the one or more flavonoids are provided as a synthetic compound or a mixture of synthetic compounds, or as a chemically modified compound or mixture of chemically modified compounds.

Carrier Oil

In embodiments of the present invention, the composition comprises a carrier oil. The characteristics of the carrier oil are not limited except that the oil must not interact with the other components of the composition. Preferably, the oil is an oil which is defined as GRAS (generally recognised as safe for human consumption). In some embodiments of the present invention, the composition comprises a carrier oil is selected from: hemp seed oil, coconut oil, rape seed oil, medium-chain triglyceride (MCT) oil, olive oil, cranberry oil and vegetable oil, or a mixture of two or more thereof. In other embodiments, the carrier oil is a mixture of hemp seed oil and coconut oil. In other embodiments, the carrier oil is a mixture of rape seed oil and coconut oil.

As such, in some embodiments the carrier oil dilutes the concentration of the other components of the composition. However, it is to be appreciated that the ratio of CBD:CBDA and the ratio of the other components to the cannabinoids remains the same following such dilution.

In some embodiments, the cannabinoid:oil ratio is 1:2 to 1:3500 w/w in the composition. In preferred variants of this embodiment, the cannabinoid:oil ratio is 1:2 to 1:2000 w/w, 1:3 to 1:500 w/w, 1:3 to 1:200 w/w, 1:3 to 1:5 w/w, 1:5 to 1:500 w/w, 1:20 to 1:100 w/w or 1:50 to 1:100 w/w.

It is to be understood that, in some embodiments the composition is provided without a carrier oil. In these embodiments, the composition is provided in a concentrated form which may not be generally safe for human consumption due to the concentration of cannabinoid. In these embodiments the concentrated form of the composition is provided to a business user or end user for mixing with carrier oil before use. Thus in some embodiments, there is provided the composition in concentrated or isolate form and a suitable amount of carrier oil (in a receptacle for example). In these embodiments, the end user combines the carrier oil and the concentrate or isolate to provide a final, dilute product in which the concentration of cannabinoid and other components are suitable for human consumption.

Nature of the Formulation

In embodiments of the present invention, the composition is produced as a concentrate, an isolate, a co-crystallised isolate, an oil, a liposomal formulation, a food bar, a savoury or sweet food such as a nutritional food bar or chocolate, a beverage, a pharmaceutical composition, or it may be nano-encapsulated, or emulsified or formed into an aerosol. In other embodiments of the present invention, the composition is produced as a tablet, capsule, cream, ointment, pessary or suppository. In other embodiments, the composition can constitute less than 10 wt % or less than 5 wt % of a food bar or beverage.

Use

The composition of the present invention is used in the treatment of inflammation or a disorder associated with inflammation. In such a clinical setting, the composition of the present invention is preferably provided in a pharmaceutical-style formulation, more preferably the formulation comprises a pharmaceutical excipient and/or diluent.

In alternative embodiments, the composition of the present invention is used for the treatment of inflammation or a disorder associated with inflammation of a human individual or animal.

However, it is also to be understood that in alternative embodiments, the composition of the present invention is used in order to alleviate inflammation of a sub-clinical nature. In such alternative embodiments, the composition of the present invention is preferably provided in a food or beverage product, preferably in a chewing gum, lozenge or tincture, or a tablet or capsule which is dissolvable in a beverage.

In further embodiments, the composition is used to prevent inflammation or a disorder associated with inflammation. For example, in some embodiments, the composition is administered or consumed in advance of a period when inflammation or a disorder associated with inflammation is otherwise expected.

Administration

In some embodiments the composition of the present invention is administered by ingestion (for example from drinking, eating or consuming capsules or tablets), buccal or nasal absorption, oral absorption (for example from tinctures, lozenges or chewing gum), inhalation (for example, from vaping, a dry powder inhaler or a nebuliser), systemic injection, or transdermal or topical absorption (for example from patches, creams, ointments, pessaries and suppositories). In some embodiments of the present invention, the composition is administered through a dropper or spray. In other embodiments of the present invention, the composition is administered intravenously. In other embodiments of the present invention, the composition is administered intrauterinally.

In some embodiments, the daily dosage is varied to suit the individual human or animal (for example to take into account the medical condition being treated). In some embodiments, the frequency of use or administration determines the daily dosage of the composition of the present invention. In some embodiments, the composition of the present invention is delivered as a single dose per day. In other embodiments, the composition of the present invention is delivered in multiple, smaller doses per day up to a total daily dosage. In certain embodiments, the composition of the present invention is delivered by microdosing or by way of a bolus.

In some embodiments of the present invention, a composition comprising 1-30% w/w CBD and CBDA, administered through a dropper, delivers 0.8-24 mg CBD and CBDA, per drop. In some embodiments, an individual human or animal requiring a daily dose of 12 mg CBD and CBDA is administered over the course of 24 hours with one drop of a composition comprising 15% w/w CBD and CBDA, or a mixture thereof, per day. In alternative embodiments, an individual human or animal requiring a daily dose of 12 mg CBD and CBDA is administered over the course of 24 hours with two drops of a composition comprising 7.5% w/w CBD and CBDA.

In alternative embodiments, a composition comprising 1-30% w/w CBD and CBDA, administered through a spray, delivers 1.4-42 mg CBD and CBDA per spray. In some embodiments, an individual human or animal requiring a daily dose of 30 mg CBD and CBDA is administered over the course of 24 hours with one spray of a composition comprising 21.4% w/w CBD and CBDA per day. In alternative embodiments, an individual human or animal requiring a daily dose of 30 mg CBD and CBDA is administered over the course of 24 hours with four sprays of a composition comprising 5.35% w/w CBD and CBDA.

Inflammation and Disorders Associated with Inflammation

In embodiments of the present invention, the composition is for use in the treatment or prevention of inflammation or a disorder associated with inflammation.

Inflammation is characterized by five classical symptoms, including heat, pain, redness, swelling and loss of function. Inflammation can be classified as either acute or chronic. Acute inflammation is typically short-term, appearing within a few minutes or hours of exposure to the cause of inflammation (such as bacterial pathogens or injury) and begins to resolve upon the removal of the cause of inflammation. By contrast, chronic inflammation is longer term, sometimes with a delayed onset and often persists for many months, or even years. Chronic inflammation can arise in response to non-degradable pathogens, viral infection, persistent foreign bodies, or autoimmune reactions.

In some embodiments, the inflammation or disorder associated with inflammation is associated with increased Pattern Recognition Receptor (PRR) activation or increased interleukin-6 (IL-6) production. This means inflammation that is present in an individual who has PRR activation or IL-6 production that is higher than in the individual prior to the inflammation arising and/or who has PRR expression or IL-6 production that is higher than average for the population (e.g. higher than average for other members of the population of the same age and gender).

Pattern recognition receptors (PRRs) are proteins that are predominantly expressed by innate immune cells (such as dendritic cells, macrophages, monocytes and neutrophils). PRRs can be membrane-bound or cytoplasmic, and function to recognise two main classes of molecule, in particular pathogen-associated molecular patterns (PAMPs), which are associated with pathogens such as bacteria, and damage-associated molecular patterns (DAMPs), which are associated with cell components which are released as a result of cell damage or death. Toll-like Receptors (TLRs) are transmembrane proteins, which are a subclass of membrane-bound PRRs. Toll-like Receptor 2 (TLR2) and Toll-like Receptor 4 (TLR4) are examples of TLRs that, when stimulated, activate intracellular signalling pathways that in turn activate the innate immune response, leading to the production of cytokines, such as interleukin-6 (IL-6) in some cases. TLR4 expression can also be increased in response to environmental factors, such as pollution (Williams et al. 2007, Biomarker Insights; 2: 226-240).

Interleukin-6 (IL-6) is a cytokine with pleiotropic (or multiple) effects. IL-6 is produced in response to infections and tissue injuries, and stimulates acute phase responses, haematopoiesis and immune reactions, including the stimulation of antibody production and T-cell development. Due to its pleiotropic effects, dysregulated and/or continual production of IL-6 can contribute to the onset and development of numerous disorders, including autoimmune and chronic inflammatory diseases.

In one embodiment, the levels of IL-6 can be measured as discussed in the Example below. The methods disclosed herein are also referred to in De Barros et al. (2018; Journal of Equine Veterinary Science; 66, p. 148; meeting abstract) and Wonfor et al. (2017; Theriogenology; 102, p. 174-182) In alternative embodiments, both TLR and IL-6 levels or expression can be measured using any method known to the skilled person, including antibody-dependent methods such as western blotting, protein immunoprecipitation and immunohistochemistry, spectrometry methods, reverse transcription-PCR and quantitative PCR.

Examples of disorders associated with inflammation, and in particular are associated with increased IL-6 production, include rheumatoid arthritis, juvenile idiopathic arthritis, ankalosing spondylitis, takayasu arteritis, giant cell arteritis, atherosclerosis, intracerebral haemorrhage, Behcet's disease, polymyalgia rheumatic, familial Mediterranean fever, thyroid eye disease, systemic lupus erythematosus, endometritis, inflammatory bowel disease, equine gastric ulcer syndrome, acute respiratory distress syndrome, asthma, cystitis, ulcerative colitis, Crohn's disease, cancer such as Castleman disease, multiple myeloma, prostate cancer, breast cancer, oral cancer, metastatic cancer, pancreatic cancer, lung cancer, liver cancer, bladder cancer, Waldenstroms macroglobulinaemia, vulvar cancer and kaposis sarcoma, infectious diseases such as COVID-19, encephalitis, enterovirus 71 and hand foot and mouth disease, severe asthma, psychiatric illnesses such as depression, schizophrenia and diseases related to epigenetic modification, neurological diseases such as neuromyelitis optica, multiple sclerosis and Alzheimer's disease, and dermatological diseases such as eczema, atopic dermatitis, psoriasis and acne, diabetes, obesity and ageing.

Species

In embodiments of the present invention, the composition is for use in the treatment or prevention of inflammation or a disorder associated with inflammation in mammals such as camels, cats, cows, dogs, horses, humans, mice, pigs, rats and sheep.

### Example 1—Bovine Endometrial Explant Culture and IL-6, PGF_{2α} and PGE₂ ELISA Analysis—Hemp Extract or Purified CBG/CBD Compound

In the following Example, the terms “crude hemp extract” and “hemp extract” refer to “Fraction 2”, which is further described in Example 2 below. Table 1 summarizes the cannabinoid content of Fraction 2.

Hemp Fraction Preparation:

Crude hemp extract was solubilised through addition of DMSO allowing a 10 mg/ml (w/v) stock solution to be prepared. The resulting stock solution was refrigerated prior to downstream use in bovine endometrial explant culture.

CBG/CBD Compound Purification

Purified CGB/CBD compound (50 μg/mL CBG: 20 μg/mL CBD) was provided by TTS Pharma by extraction with ethanol.

Bovine Endometrial Explant Culture:

Tissue Collection:

Uteri were collected from freshly slaughtered beef cattle in a local Welsh abattoir. Female tracts were identified and uteri assessed prior to removal of all surrounding fat and tissue. If suitable the cervix was located and the tract cut off posterior to this. Ovaries were assessed and their stage recorded following examination of the corpus luteum. The removed uteri were placed into individual bags and stored on ice for processing upon arrival in the lab.

Endometrial Tissue Extraction:
1. Work bench was thoroughly cleaned with 70% IMS and covered in sterilised foil
2. Individual uteri were placed onto the foil and their external surface cleaned with 70% IMS
3. Uterine horn was cut ipsilaterally to the ovary with care taken not to damage the underlying endometrium
4. An 8 mm biopsy punch was utilised in order to collect tissue from intercaruncular areas—scissors were used in order to dissect the endometrium away from underlying tissue with successful dissection identified though biopsies appearing 2 mm thick and translucent

Each extracted explant was stored at 37° C. in RPMI 1640 media supplemented with:
50 IU/ml penicillin
50 μg/ml streptomycin
2.5 μg/ml amphotericin B

This process was repeated for each uterus used in the experiment (n=3) with total number of explants required for each replicate calculated prior to dissection.

Explant Culture Set-Up:

All work conducted from this point was under aseptic conditions within a laminar flow hood.
1. Explants were removed from incubation and washed twice with sterile Hanks Buffered Saline Solution (HBSS)
2. Each explant was placed into an individual well of 6-well plates (see “plate layouts”)
3. Each explant was covered with 3 ml of the corresponding pre-controls/treatments (see “pre-controls/treatments”)

Each control/treatment was tested in technical duplicate (n=2) and biological triplicate (n=3)

Culture media—RPMI 1640 media supplemented with:
50 IU/ml penicillin
50 μg/ml streptomycin
2.5 μg/ml amphotericin B
10% Fetal bovine serum (FBS)

Pre-Controls/Treatments:

| Sample name | Composition |
|---|---|
| Control 1 | Culture media only |
| Control 2 | 10 μg/ml hemp extract or CBG/CBD compound |
| Control 3 | 25 μg/ml hemp extract or CBG/CBD compound |
| Control 4 | 50 μg/ml hemp extract or CBG/CBD compound |
| Treatment 1 | Culture media only |
| Treatment 2 | 10 μg/ml hemp extract or CBG/CBD compound |
| Treatment 3 | 25 μg/ml hemp extract or CBG/CBD compound |
| Treatment 4 | 50 μg/ml hemp extract or CBG/CBD compound |

Plate Layouts:

Plate layouts remained consistent for each biological replicate (n=3).

Plate 1

| Control 1 | Control 2 | Control 3 |
|---|---|---|
| Control 1 (replicate) | Control 2 (replicate) | Control 3 (replicate) |

Plate 2

| Control 4 | Treatment 1 | Treatment 2 |
|---|---|---|
| Control 4 (replicate) | Treatment 1 (replicate) | Treatment 2 (replicate) |

Plate 3

| Treatment 3 | Treatment 4 | — |
|---|---|---|
| Treatment 3 (replicate) | Treatment 4 (replicate) | — |

Following addition of pre-controls/treatments, plates were labelled with the date and time of culture start and placed in an incubator at 37° C. with 5% CO₂. After ˜16 hours media was removed and replaced with controls/treatments—those requiring LPS were now included (see “Treatments” below).

Treatments:

| Sample name | Composition |
|---|---|
| Control 1 | Culture media only |
| Control 2 | 10 μg/ml hemp extract or CBG/CBD compound |
| Control 3 | 25 μg/ml hemp extract or CBG/CBD compound |
| Control 4 | 50 μg/ml hemp extract or CBG/CBD compound |
| Treatment 1 | Culture media + 500 ng/ml LPS |
| Treatment 2 | 10 μg/ml hemp extract or CBG/CBD compound + |
| | 500 ng/ml LPS |
| Treatment 3 | 25 μg/ml hemp extract or CBG/CBD compound + |
| | 500 ng/ml LPS |
| Treatment 4 | 50 μg/ml hemp extract or CBG/CBD compound + |
| | 500 ng/ml LPS |

Supernatants were collected from each well at 6 and 24-hour timepoints and stored at −20° C. for downstream analysis.

Inflammatory Marker (IL-6, PGF_{2α} and PGE₂) ELISA:

Stored supernatants were analysed through a Bovine IL6 ELISA kit (Invitrogen®-ESS0029), following the manufacturer's instructions (below). Alternatively, the stored supernatants were analysed through a prostaglandin F2 alpha (PGF_{2α}) ELISA kit (Enzo Life Sciences, ADI-900-069) or prostaglandin E₂ (PGE₂) ELISA kit (Enzo Life Sciences, ADI-900-001), as per the manufacturer's instructions. Each supernatant was run in duplicate and biological replicates were combined for downstream statistical analysis.

ELISA Reagent Kit Buffers:

D-PBS: 0.008M sodium phosphate, 0.002M potassium phosphate, 0.14M sodium chloride, 0.01M potassium chloride, pH 7.4, 0.2 μm filtered (e.g., Thermo Scientific® BupH® Modified Dulbecco's Phosphate Buffered Saline Packs, Product No. 28374)
Carbonate-bicarbonate Buffer: 0.2M sodium carbonate-bicarbonate buffer, pH 9.4, 0.2 μm filtered (e.g., BupH Carbonate/Bicarbonate Buffer, Product No. 28382)
Blocking Buffer: 4% BSA, 5% sucrose in D-PBS, 0.2 μm filtered OR ELISA Blocker Blocking Buffer, Product No. N502
Reagent Diluent: 4% BSA in D-PBS (pH 7.4), 0.2 μm filtered
Wash Buffer: 0.05% Tween™-20 Detergent (e.g., 0.5% Thermo Scientific® Surfact-Amps® 20 Detergent Solution,

Product No. 28320) in D-PBS, pH 7.4 OR ELISA Wash Buffer (30×), Product No. N503 Note: Mix new solution daily.

Assay Protocol:

Kit components are titered to give optimal results using the Bovine IL-6 ELISA Reagent Kit Protocol for cell culture supernatants. Any change, including component concentration, volumes, incubation times or temperatures, buffer content or number of wash steps may significantly affect the ELISA results and require optimization to give the best results.

Note: Allow all reagents and buffers to equilibrate to room temperature (22-25° C.) before use. Thaw one aliquot of coating and detecting antibody for each plate. Do not use a water bath.

A. Plate Preparation
1. Dilute the Coating Antibody 1:100 in carbonate-bicarbonate buffer by adding 110 μL Coating Antibody to 10.89 mL of carbonate-bicarbonate buffer.
2. Add 100 μL of diluted Coating Antibody to each well. Cover plate with plate sealer and incubate overnight at room temperature.
3. Aspirate Coating Antibody solution and add 300 μL of Blocking Buffer to each well. Cover plate with plate sealer and incubate for 1 hour at room temperature.
4. Aspirate Blocking Buffer and proceed to assay or allow to dry overnight at room temperature. When sealed with dessicant, plates can be stored at 2-8° C. for 6 months.

B. Assay Procedure
5. Reconstitute standard with Reagent Diluent with volume stated on vial label. The concentration of the reconstituted standard is 10,000 μg/mL.
6. Dilute reconstituted standard 1:2 in Reagent Diluent to prepare top Standard (5000 μg/mL). Using Reagent Diluent, prepare 1:2 serial dilutions of top Standard and dilute any supernatant expected to read above the top standard. Add 100 μL of sample or Standard to each well. Cover plate with plate sealer and incubate for 1 hour at room temperature with moderate shaking.
7. Aspirate and wash three times with Wash Buffer using 300 μL per well.
8. Dilute the Detection Antibody 1:100 in Reagent Diluent by adding 110 μL of Detection Antibody to 10.89 mL of Reagent Diluent.
9. Add 100 μL of Detection Antibody to each well. Cover plate with plate sealer and incubate for 1 hour at room temperature with moderate shaking.
10. Aspirate and wash three times with Wash Buffer, using 300 μL per well.
11. Dilute Streptavidin-HRP 1:400 in Reagent Diluent by adding 30 μL of Streptavidin-HRP to 12 mL of Reagent Diluent.
12. Add 100 μL of diluted Streptavidin-HRP reagent to each well. Cover plate with plate sealer and incubate for 30 minutes at room temperature with moderate shaking.
13. Aspirate and wash three times with Wash Buffer, using 300 μL per well.
14. Add 100 μL of Substrate Solution to each well. Cover plate with plate sealer and incubate in the dark for 20 minutes at room temperature.
15. Stop the reaction by adding 100 μL of Stop Solution to each well.
16. Measure the absorbance at A450 minus A550.

C. Absorbance Measurement
17. Measure absorbance on an ELISA plate reader set at 450 nm and 550 nm. Subtract 550 nm values from 450 nm values to correct for optical imperfections in the microplate. If an absorbance at 550 nm is not available, measure the absorbance at 450 nm only.

Note: When the 550 nm measurement is omitted, absorbance values will be higher.

Note: Evaluate the plate within 30 minutes of stopping the reaction.

Calculation of Results:
The standard curve is used to determine inflammatory marker amount in an unknown sample. Generate the standard curve by plotting the average absorbance obtained for each Standard concentration on the vertical (Y) axis vs. the corresponding inflammatory marker concentration (pg/mL) on the horizontal (X) axis.
Calculate results using graph paper or curve-fitting statistical software. Determine the inflammatory marker amount in each sample by interpolating from the absorbance value (Y-axis) to inflammatory marker concentration (X-axis) using the standard curve.
If the test sample was diluted, multiply the interpolated value obtained from the standard curve by the dilution factor to calculate pg/mL of inflammatory marker in the sample.
The values obtained were then corrected for explant weight.
Absorbance values obtained for duplicates should be within 10% of the mean value. Carefully consider duplicate values that differ from the mean by greater than 10%.

Resulting data was analysed and calibration graphs created. SPSS statistical analysis was used in order to look at significant differences between treatment and control groups. Specifically, a Tukeys post-hoc test was utilised with significance assumed when P<0.05. Both inter- and intra-control samples were also utilised whilst running the inflammatory marker ELISAs allowing replicability between plates to be determined.

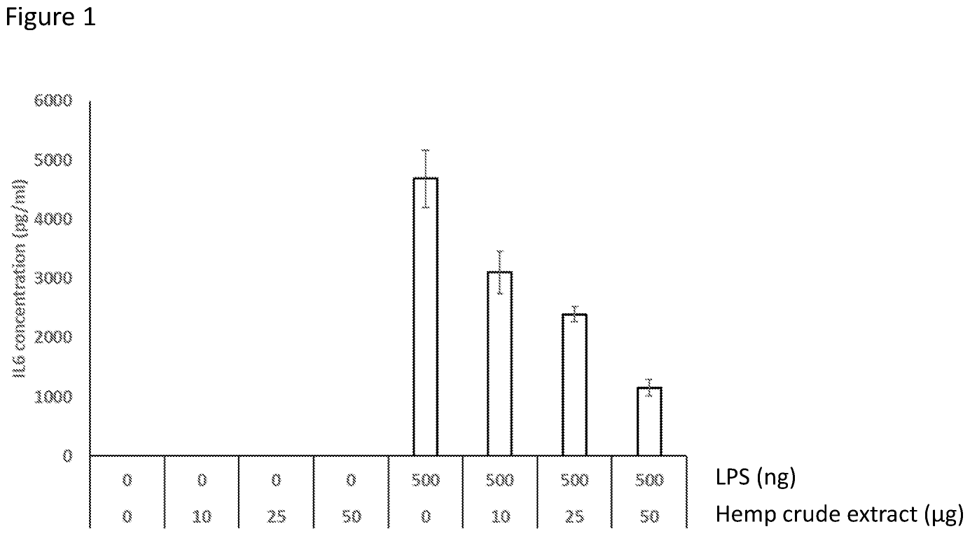

FIG. 1 shows the IL-6 concentration (pg/ml) in response to the addition of LPS and/or crude hemp extract.

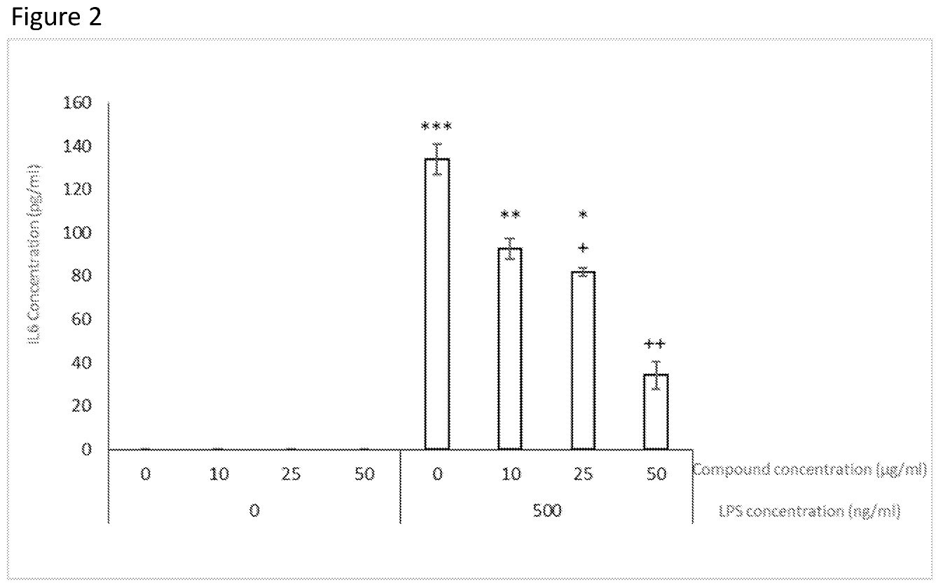

FIG. 2 (revised FIG. 1 corrected for dilutions and explant weights and with statistical analysis) shows the IL-6 concentration (pg/ml) in response to the addition of LPS and/or crude hemp extract.

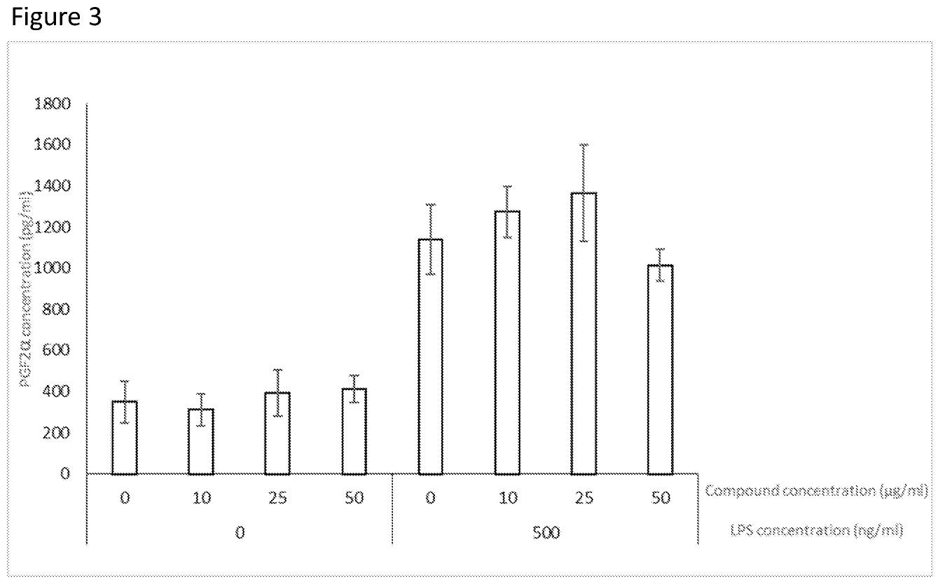

FIG. 3 shows the PGF_{2α} concentration (pg/ml) in response to the addition of LPS and/or crude hemp extract.

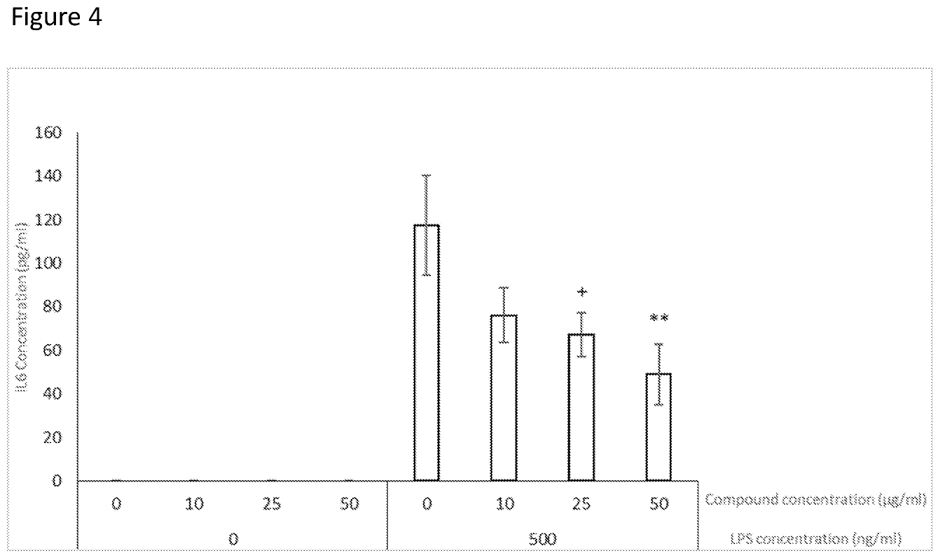

FIG. 4 (revised FIG. 1 with statistical analysis) shows the IL-6 concentration (pg/ml) in response to the addition of LPS and/or the cannabinoid combination CBG/CBD.

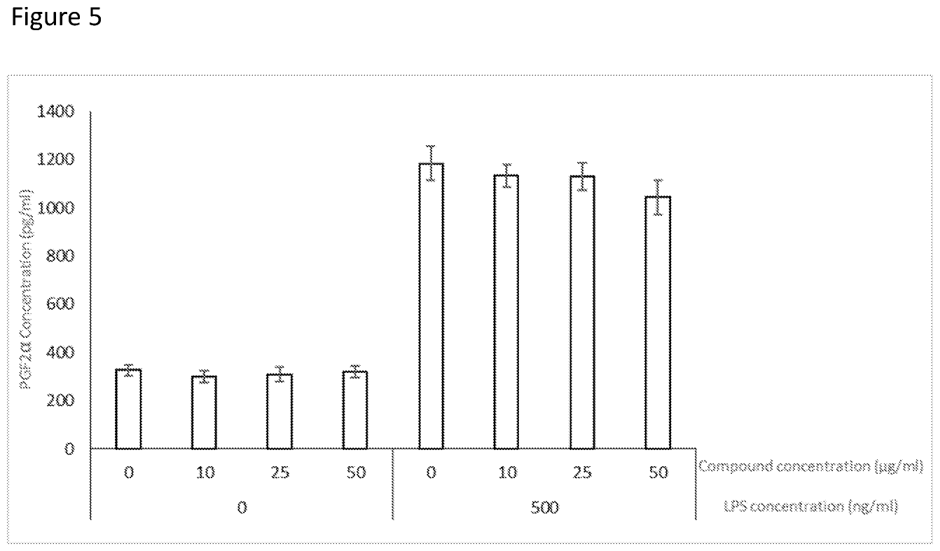

FIG. 5 shows the PGF_{2α} concentration (pg/ml) in response to the addition of LPS and/or the cannabinoid combination CBG/CBD.

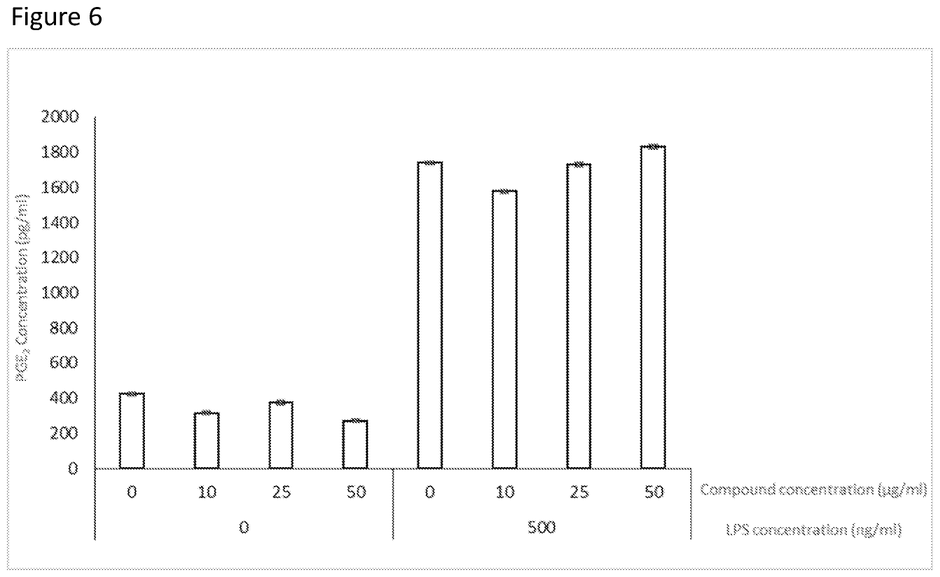

FIG. 6 shows the PGE₂ concentration (pg/ml) in response to the addition of LPS and/or the cannabinoid combination CBG/CBD.

Results:

To demonstrate that LPS alone caused an inflammatory response, 500 ng/ml LPS stimulated an increase of IL6 that was significantly greater than the control (P<0.001). Addition of crude hemp extract alone (10, 25 and 50 μg/mL) to the culture medium did not cause any inadvertent IL6 increase compared to the control. Crude hemp extract at a concentration of 50 μg/mL abrogated IL6 when added in combination with LPS to the culture medium so that concentrations were not different to that of untreated control secretion, indicating it had an anti-inflammatory effect in terms of this specific inflammatory marker. Furthermore, crude hemp extract concentrations of 25 and 50 μg/mL reduced IL6 secretion compared to that of the LPS only treated explants (P<0.05 and P<0.01 respectively).

Following the reduction in IL6 observed with treatment with the crude hemp extract the tissue culture supernatants were subsequently analysed for the response on PGF_{2α}. Addition of crude hemp at 10, 25 and 50 μg/mL in the absence of LPS did not cause an increase in PGF_{2α} when compared to the control. Stimulation of the tissue with 500 ng/mL LPS caused an increase in PGF_{2α} secretion, however subsequent increasing concentration of crude hemp extract did not cause a reduction as observed during the IL6 analysis.

Following identification of anti-inflammatory activity through crude hemp extract effects on IL6, a CBG/CBD (50 μg/mL CBG: 20 μg/mL CBD) composition provided by TTS Pharma Ltd was evaluated through the bovine explant system. The resulting tissue culture supernatants were assessed for their effects on the inflammatory markers IL6, PGF_{2α}, and PGE₂.

The effects of CBG/CBD were first investigated through IL6 following the significant reduction observed in IL6 with treatment of the crude hemp fraction. Addition of LPS caused an increase in IL6 concentration, with the addition of 50 μg/mL CBG/CBD compound reducing IL6 secretion when compared to the LPS control (P<0.01).

Tissue culture supernatant was further tested for the effects of the CBG/CBD on PGF_{2α}. Again, treatment with 10, 25 and 50 μg/mL of CBG/CBD composition did not cause an increase in the baseline PGF_{2α} concentration observed in the control. Addition of 500 ng/mL LPS increased PGF_{2α} secretion by the tissue, however treatment with CBG/CBD composition at varying concentrations did not show any reduction in PGF_{2α} levels.

Finally, the tissue culture supernatant was tested for the effects of the CBG/CBD on PGE₂. Addition of CBG/CBD composition did not cause any differences to baseline PGE₂ concentration. Stimulation with LPS showed an increase in PGE₂, however addition of CBG/CBD composition did not cause any reduction in PGE₂ levels at any of the concentrations tested.

In summary, analysis of tissue culture supernatants following pre-treatment with the cannabinoid compositions at varying concentrations and the addition of LPS demonstrate IL6 to be the primary cytokine reduced through the addition of both crude hemp extract and the CBG/CBD composition. In contrast, no response was observed for the addition of crude hemp extract or the CBG/CBD composition on the non-cytokine inflammatory markers PGF_{2α} and PGE₂. In light of this, the anti-inflammatory effects of crude hemp extract and the CBG/CBD composition are understood to target the IL-6 pathway specifically with a low possibility of adverse effects or toxicity through other inflammatory pathways including prostaglandins.

### Example 2

Extraction of hemp fibre pellets with supercritical CO₂:

Dungaro hemp pellets were extracted with a bespoke supercritical CO₂ rig comprised of two extraction vessels with a combined working volume of 10 litres. Extraction pressure was 180 bar, temperature 40° C. and density 816.1.

Fractions were collected under the following conditions:
Fraction collector 1) 120 bar, 50° C., density 510.6—green fraction, contains chlorophyll, lipids and cannabinoids;
Fraction collector 2) 80 bar, 40° C., density 219—yellow fraction, contains cannabinoids
Fraction collector 3) 50 bar, 25° C., density 113—oil fraction, contains terpenes and cannabinoids.
Fraction weights, 1) 19.3%, 2) 77.5%, 3) 3.21%

Preparation for Analysis:

Weighed 10 mg of extract and added to 5 mL 100% methanol and placed on a shaker for 1 hr. Samples were centrifuged for 15 min at 10,000 g and the supernatant was transferred to a clean container. The pellet was re-extracted with 5 mL 100% methanol and centrifuged as above. The supernatant was combined with the first supernatant fraction and this total volume was dried down in a vacuum centrifugal drier. The dried residue was resuspended in 100 μL, diluted 1:10 and analysed by LC-PDA-MSn as described below.

The analysis was carried out with a Thermo Finnigan LTQ MS® system (Thermo Electron Corporation, USA) comprising a Finnigan Surveyor PDA Plus® detector, a Finnigan LTQ® linear ion trap with ESI source and a Waters C18Nova-Pak column (60 Å, 4 μm, 3.9 mm×150 mm; WAT036975) (with guard column fitted) and autosampler.

Instrument Conditions:

The instrument is operated via the Xcalibur® software programme, the following conditions are set in the method file—
Autosampler: The sample tray is maintained at 5° C. The column is maintained at 30° C. Injection volume 10/20 μL
Pump: LC solvents: A: water with 0.1% formic acid; B: methanol with 0.1% formic acid, flow rate: 1 mL/min; gradient: 60-100% B in 20 min. Wash step: 5 min 100% B, 10 min 60% B.
PDA: scan range 240-400 nm
Mass Spectrometer conditions: tune file: chlorogenic0608. Sheath gas 30 (arbitrary units), auxiliary gas 15, sweep gas 0, capillary Temperature 320° C., spray voltage −4.0 kV/+4.8 kV, capillary voltage −1V/+45V, tube lens −68V/+110V.

Mass Spectrometer detection: scan events:
Run 1:
PDA: wavelength range 240-400 nm
MS:
Full MS positive mode,
MS2 357.00@cid35.00 [95.00-360.00] in negative mode (targeting CBDA)
MS2 315.00@cid35.00 [85.00-320.00] in positive mode (targeting THC and CBD)
Run 2:
PDA: wavelength range 240-400 nm
MS:
Full MS positive mode,
Data-dependent scan targeting most abundant ion.

Data files analysed using Qual Browser in Xcalibur®.

Results:

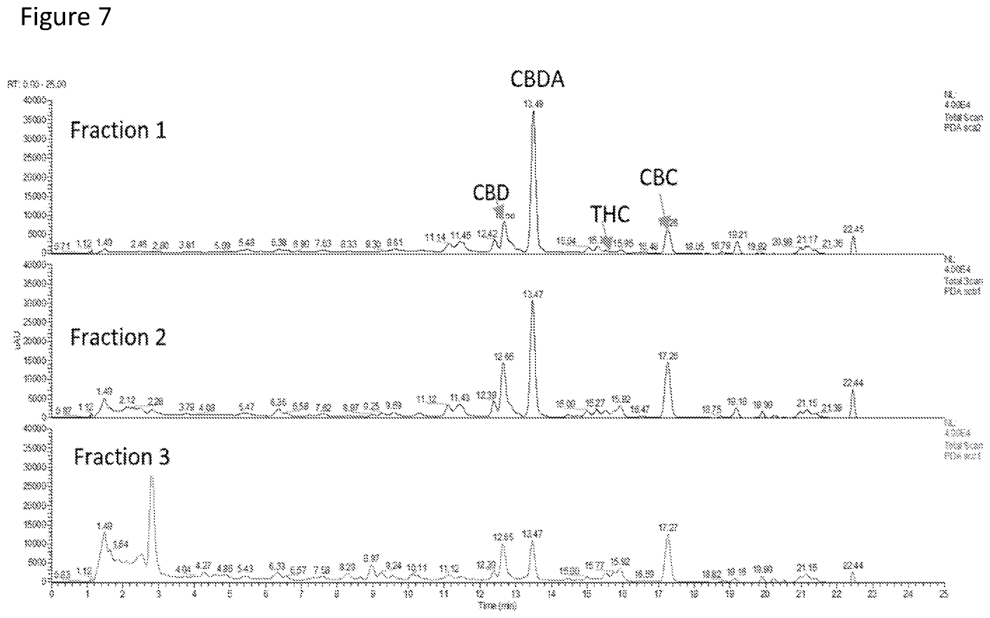

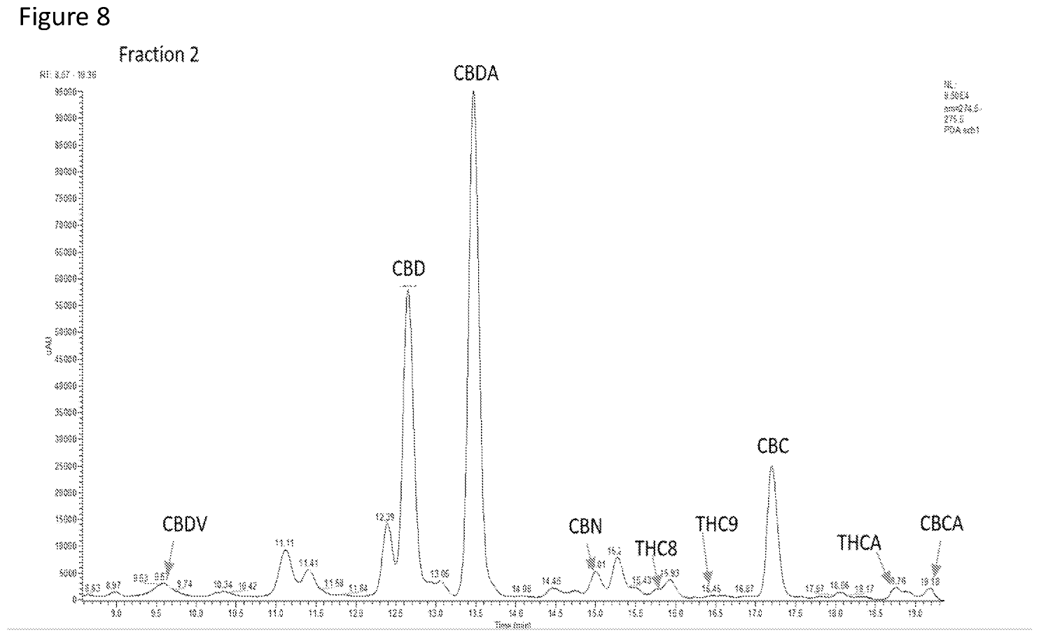

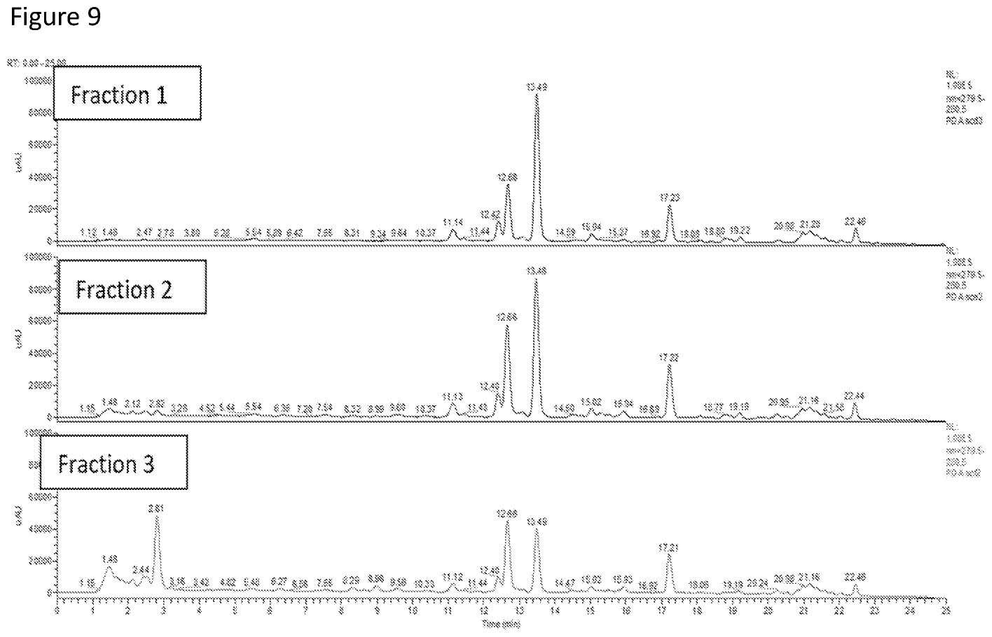

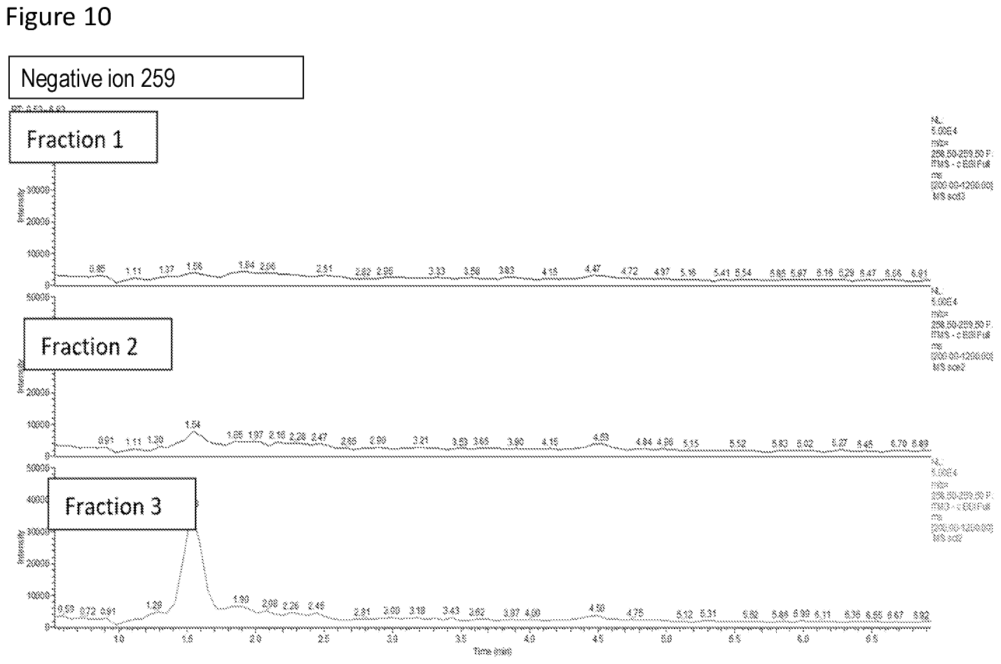

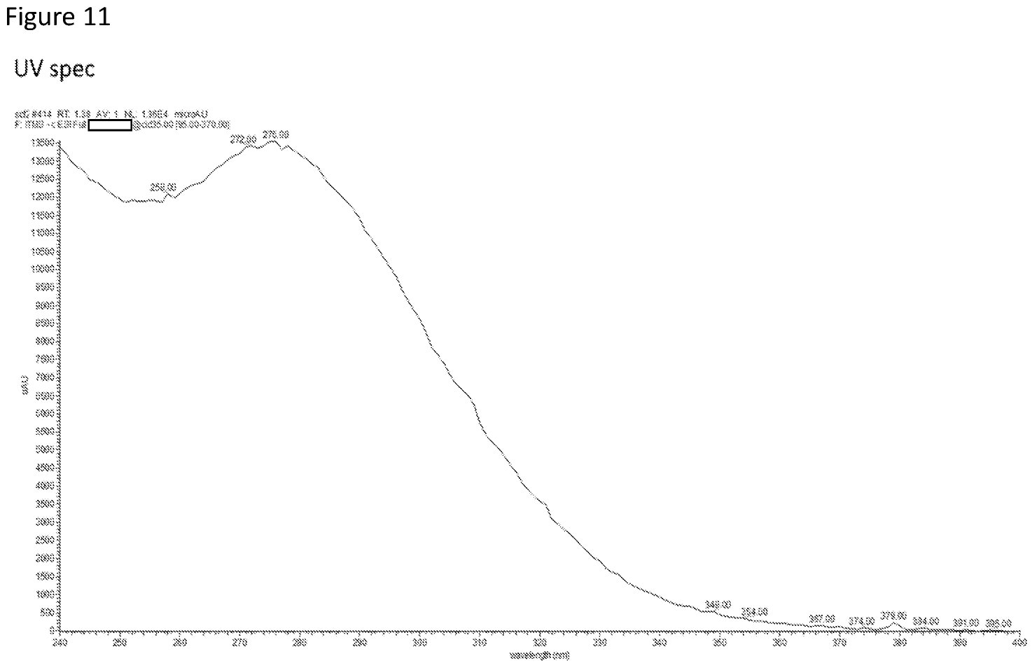

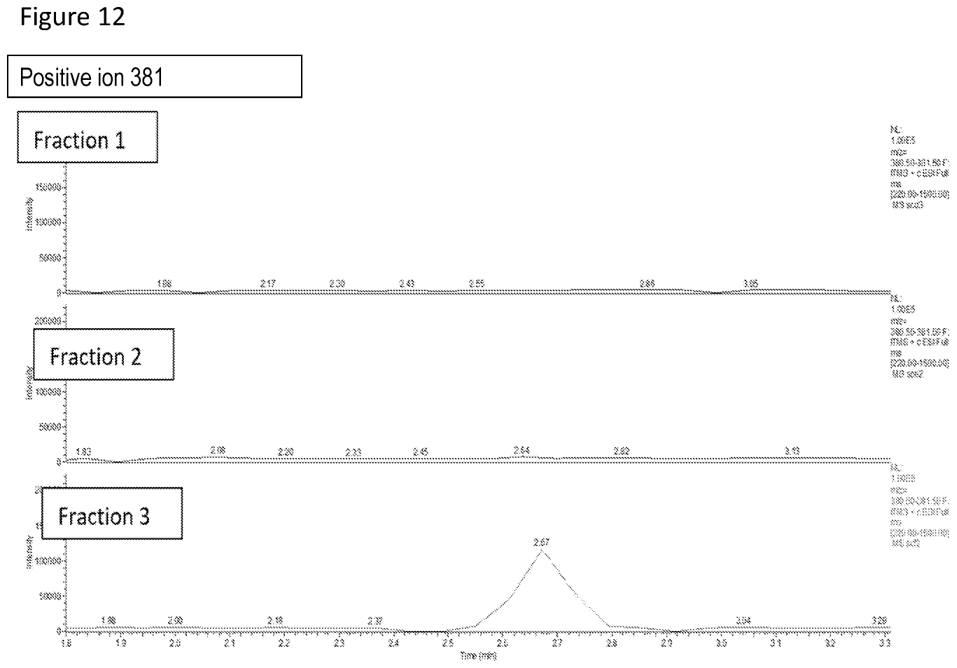

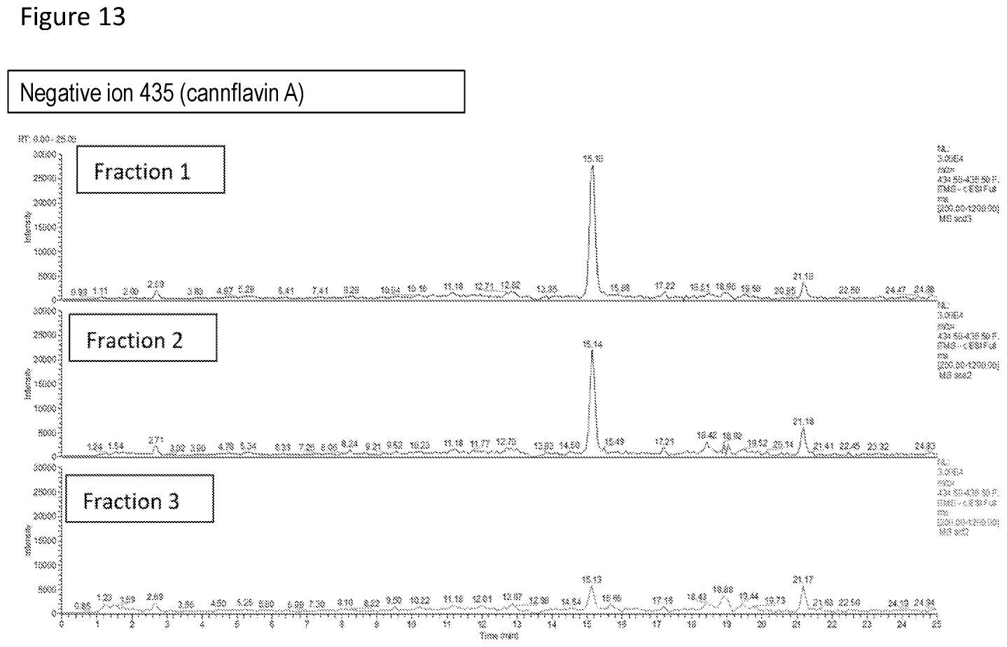

FIGS. 7 to 13 show chromatograms showing the analysis of the different fractions. Fraction 1 contained lipids and chlorophylls and fraction 3 contained terpenes. All fractions contained cannabinoids but most material went into fraction 2 (77.5% of total product weight). A relatively high content of CBC was also detected in these fractions.

Table 1 below summarizes the cannabinoid content of the fractions.

% Cannabinoids in Supercritical CO₂ fractions

| | Fraction 1 | Fraction 2 | Fraction 3 |
|---|---|---|---|
| CBDV | 0.430 | 0.427 | 0.447 |
| CBD | 7.299 | 9.795 | 8.676 |
| CBDA | 2.128 | 1.704 | 0.934 |
| CBN | 0.055 | 0.069 | 0.038 |
| THC8 | 0.257 | 0.240 | 0.162 |
| THC9 | 0.185 | 0.171 | 0.131 |
| CBC | 0.451 | 0.507 | 0.376 |
| THCA | 0.041 | 0.040 | 0.021 |
| CBCA | 0.024 | 0.018 | 0.009 |

1. A method of treating or preventing inflammation, a disorder associated with inflammation, or an IL-6 related disorder, comprising administering a composition comprising cannabidiol (CBD) and one or more cannabinoids selected from cannabidiolic acid (CBDA), cannabigerol (CBG), and cannabigerolic acid (CBGA) to a patient in need thereof.

2. The method according to claim 1, wherein the inflammation or disorder associated with inflammation is associated with increased Pattern Recognition Receptor (PRR) activation or increased interleukin-6 (IL-6) production.

3. The method according to claim 2, wherein the Pattern Recognition Receptor (PRR) is a Toll-like Receptor.

4. The method according to claim 1, wherein the inflammation or disorder associated with inflammation is selected from rheumatoid arthritis, juvenile idiopathic arthritis, ankalosing spondylitis, takayasu arteritis, giant cell arteritis, atherosclerosis, intracerebral haemorrhage, Behcet's disease, polymyalgia rheumatic, familial Mediterranean fever, thyroid eye disease, systemic lupus erythematosus, endometritis, inflammatory bowel disease, equine gastric ulcer syndrome, acute respiratory distress syndrome, asthma, cystitis, ulcerative colitis, Crohn's disease, cancer such as Castleman disease, multiple myeloma, prostate cancer, breast cancer, oral cancer, metastatic cancer, pancreatic cancer, lung cancer, liver cancer, bladder cancer, Waldenstroms macroglobulinaemia, vulvar cancer and kaposis sarcoma, infectious diseases such as COVID-19, encephalitis, enterovirus 71 and hand foot and mouth disease, severe asthma, psychiatric illnesses such as depression, schizophrenia and diseases related to epigenetic modification, and neurological diseases such as neuromyelitis optica, multiple sclerosis and Alzheimer's disease, and dermatological diseases such as eczema, atopic dermatitis, psoriasis and acne, diabetes, obesity and ageing.

5. The method according to claim 4, wherein the inflammatory bowel disease is selected from ulcerative colitis and Crohn's disease.

6. The method according to claim 1, wherein the composition further comprises at least one terpene, preferably wherein the terpene:cannabinoid ratio by weight is from 1:3 to 1:60 w/w.

7. The method according to claim 6, wherein the at least one terpene is terpinolene.

8. The method according to claim 6, wherein the at least one terpene further comprises alpha-humulene, beta-caryophyllene, alpha-pinene, beta-pinene, terpinolene, beta-myrcene and/or an ocimene isomer.

9. The method according to claim 6, wherein the at least one terpene is present at a concentration of 0.01-10% w/w, preferably 0.1-6% w/w, more preferably 0.5-3% w/w.

10. The method according to claim 1, wherein the total concentration of CBD and CBDA is 0.03-30% w/w, preferably 1-25% w/w, more preferably 5-25% w/w.

11. The method according to claim 1, wherein the total cannabinoid concentration is 0.03-30% w/w, preferably 1-25% w/w, more preferably 5-25% w/w.

12. The method according to claim 1, wherein the composition further comprises cannabidivarin (CBDV).

13. The method according to claim 12, wherein the CBDV:CBD ratio by weight is 1:10 to 1:30 w/w, preferably 1:15 to 1:25 w/w, more preferably 1:20 to 1:25 w/w.

14. The method according to claim 1, wherein the CBDA:CBD ratio by weight is from 1:2 to 1:20 w/w, preferably 1:2 to 1:15 w/w, more preferably 1:3 to 1:10 w/w, more preferably 1:4 to 1:6 w/w.

15. The method according to claim 1, wherein the composition further comprises an oil.

16. The method according to claim 15, wherein the cannabinoid:oil ratio is 1:2 to 1:3500 w/w.

17. The method according to claim 15, wherein the oil is present at 60-99.96% w/w.

18. The method according to claim 15, wherein the oil is selected from hemp seed oil, rape seed oil, coconut oil and MCT oil, or a mixture or two or more oils selected from hemp seed oil, rape seed oil, coconut oil and MCT oil.

19. A method of treating or preventing inflammation or a disorder associated with inflammation, comprising administering a food or beverage product comprising a composition according to claim 1 to a subject in need thereof.

20. A method of treating or preventing a non-clinical inflammation, comprising administering a composition according to claim 1 to a subject in need thereof.
